(19) United States
(12) Patent Application Publication
(10) Pub. No.: US 2020/0164211 A1
(43) Pub. Date: May 28, 2020

(54) LOWERING BLOOD PRESSURE BY STIMULATING BRODMANN AREA 25

(71) Applicant: Case Western Reserve University, Cleveland, OH (US)

(72) Inventors: Nuria Lacuey-Lecumberri, Cleveland, OH (US); Samden Lhatoo, Cleveland, OH (US)

(21) Appl. No.: 16/200,694

(22) Filed: Nov. 27, 2018

(51) Int. Cl. A61N 1/36; A61N 1/05

(52) U.S. Cl. CPC A61N 1/36117; A61B 5/021; A61N 1/36139; A61N 1/0531

(57) ABSTRACT

The present disclosure relates to blood pressure control. A method for blood pressure control can include receiving, by a controller comprising a processor, an input indicating a requirement for blood pressure reduction and signaling, by the controller, a stimulator to generate a stimulation waveform with parameters configured for the blood pressure reduction. The method also includes generating, by the stimulator, the stimulation waveform with the parameters configured for the blood pressure reduction. The method further includes applying, by at least one electrode located proximal to Brodmann area 25, the stimulation waveform proximal to Brodmann area 25 to reduce the blood pressure.

### TECHNICAL FIELD

The present disclosure relates generally to blood pressure control and, more specifically, to systems and methods for lowering blood pressure by stimulating Brodmann area 25.

### BACKGROUND

Resistant hypertension is a condition in which a patient's blood pressure remains high despite efforts to lower the patient's blood pressure. According to one definition, a patient is considered to have resistant hypertension when three antihypertensive agents of different classes are used together and relief is not seen. Patients with resistant hypertension have a higher risk of strokes and heart attacks than those whose blood pressure can be controlled and people who do not have high blood pressure. Additionally, patients with resistant hypertension can also have a higher risk of heart failure, vision loss, renal failure, or other maladies.

### SUMMARY

The present describes systems and methods for lowering blood pressure by stimulating Brodmann area 25.

In an aspect, the present disclosure includes a method for blood pressure control. The method includes receiving, by a controller comprising a processor, an input indicating a requirement for blood pressure reduction and signaling, by the controller, a stimulator to generate a stimulation waveform with parameters configured for the blood pressure reduction. The method also includes generating, by the stimulator, the stimulation waveform with the parameters configured for the blood pressure reduction; and applying, by at least one electrode located proximal to Brodmann area 25, the stimulation waveform to Brodmann area 25 to reduce the blood pressure.

In another aspect, the present disclosure includes a controller that can be used for blood pressure control. The controller includes a memory storing instructions; and a processor configured to access the memory and execute the instructions to: receive an input indicating a requirement for blood pressure reduction; and signal a stimulator to generate a stimulation waveform to be delivered by at least one electrode located proximal to Brodmann area 25, wherein the stimulation waveform is configured to be delivered to Brodmann area 25 by the at least one electrode to reduce the blood pressure.

In a further aspect, the present disclosure includes a closed loop system for blood pressure management. The system includes at least one sensor configured to detect blood pressure. The system also includes a controller configured to: receive the blood pressure from the at least one sensor; determine whether a value associated with the blood pressure from the at least one sensor exceeds a threshold value; and when the value associated with the blood pressure for the at least one sensor exceeds the threshold value, signal a stimulator to generate a stimulation waveform. The system also includes the stimulator configured to generate the stimulation waveform; and at least one electrode configured to be located proximal to Brodmann area 25 and to deliver the stimulation waveform to Brodmann area 25 to reduce the blood pressure.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a diagram showing a system that can be used for lowering blood pressure by stimulating Brodmann area 25 in accordance with an aspect of the present disclosure;

FIG. 2 is a diagram showing an example of the controller in FIG. 1;

FIG. 3 is a process flow diagram illustrating a method for lowering blood pressure by stimulating Brodmann area 25 according to another aspect of the present disclosure;

FIG. 4 is a process flow diagram illustrating an example method for deciding whether to generate the input in FIG. 3;

FIGS. 5-8 illustrate stimulation induced cardiovascular changes in patient 7 (stimulation at 50 Hz, 0.2 ms, and 1 to 9 mA);

FIGS. 9-12 illustrate stimulation induced cardiovascular changes in patient 8 (stimulation at 50 Hz, 0.2 ms, and 1 to 10 mA); and

FIGS. 13-16 illustrate stimulation induced cardiovascular changes in patient 9 with impaired autonomic responses (stimulation at 50 Hz, 0.2 ms, and 1 to 9 mA).

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term Brodmann area 25 can refer to a narrow band in the caudal portion of the subcallosal area adjacent to the paraterminal gyrus of the cerebral cortex of the brain. The posterior parolfactory sulcus separates the paraterminal gyrus from Brodmann area 25. Rostrally, Brodmann area 25 is bound by the prefrontal area 11 of Brodmann. Brodmann area 25 can also be referred to as the subgenual area, area subgenualis, or subgenial cingulate. In other instances, Brodmann area 25 can be referred to as the mesial frontal subcallosal region

As used herein, the term “blood pressure” can refer to the pressure of circulating blood on the walls of blood vessels. Blood pressure is usually expressed in terms of the systolic pressure (maximum during one heart beat) over diastolic pressure (minimum in between two heart beats) and is measured in millimeters of mercury (mm Hg). Blood pressure can be detected non-invasively or invasively, with one or more sensors being implanted within a patient's body (e.g., intra-arterial sensors or extra-arterial sensors, like an atrial cuff sensor).

As used herein, the term “controller” can refer to a device that manages, commands, directs, and/or regulates the behavior of other devices based on an input. The input can be a feedback input (“closed loop”) or a non-feedback input (“open loop”). An example of a feedback input is a blood pressure measurement recorded after stimulation of Brodmann area 25. An example of a non-feedback input is a user input (which may be based on a blood pressure measurement, but need not be based on the blood pressure measurement). Controllers can be hardware elements that include processing and memory capabilities of a processor and/or a non-transitory memory.

As used herein, the term “proximal” can refer to a location that is near a target (e.g., Brodmann area 25). For example, a stimulation that is applied proximal to Brodmann area 25 can be applied from an electrode contacting or within Brodmann area 25, over Brodmann area 25, or near Brodmann area 25 (e.g., applied within or near the subcallosal region of the brain).

As used herein, the term “subject” can be used interchangeably with “patient” and refer to any warm-blooded organism including, but not limited to, a human being, a pig, a rat, a mouse, a bird, a dog, a cat, a goat, a sheep, a horse, a monkey, an ape, a rabbit, a cow, etc.

### II. Overview

The present disclosure relates generally to blood pressure control. High blood pressure puts an extra strain on a patient's arteries and heart. High blood pressure can be adequately controlled in many patients with one or more pharmaceutical agents. However, in patients with resistant hypertension, the patient's blood pressure remains elevated despite efforts to lower the patient's blood pressure, putting the patient at risk for stroke and heart attack, as well as many other complications. In patients with resistive hypertension, as well as many other types of hypertension, stimulation of the subcallosal region, specifically Brodmann area 25, can provide an alternative to traditional pharmaceutical therapies. As such, the present disclosure relates, more specifically, to systems and methods for lowering blood pressure by stimulating Brodmann area 25.

While not wishing to be bound by theory, it appears that the neural pathway that is related to blood pressure control extends through Brodmann area 25, making Brodmann area 25 a consistent and exquisite control site for blood pressure. Accordingly, by stimulating Brodmann area 25, the neural pathway is disrupted and, consequentially, at least the systolic blood pressure is reduced. For example, based on one or more parameters of the waveform used to stimulate Brodmann are 25, the blood pressure can be reduced by 20 points systolic or more.

### III. Systems

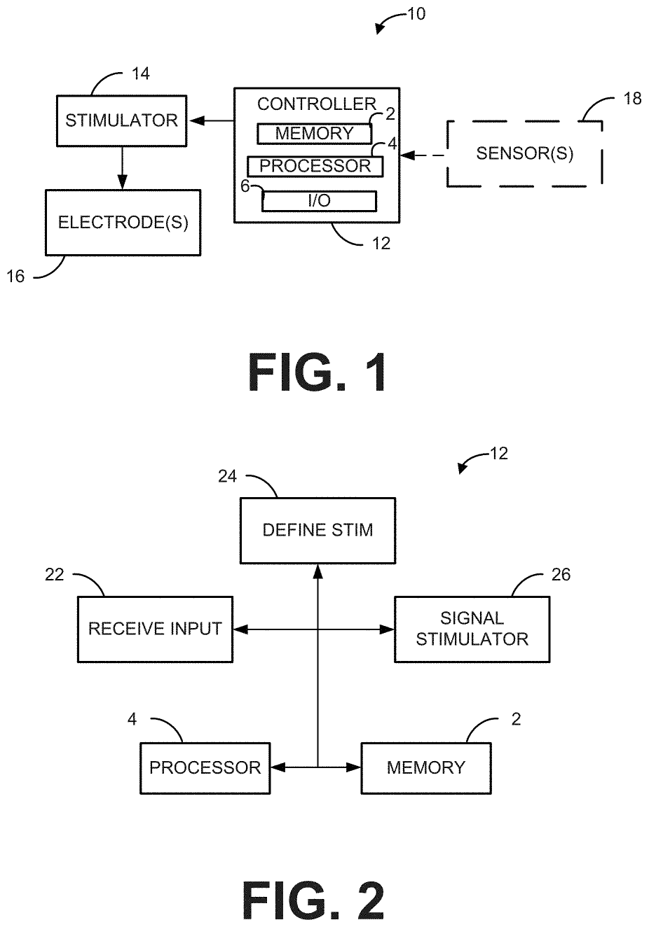

One aspect of the present disclosure can include a system 10 (FIG. 1) that can be used for lowering blood pressure by stimulating Brodmann area 25. The system 10 can include a controller 12, a stimulator 14, and one or more electrodes 16. In some instances, the system 10 can include one or more sensors 18. The system 10 may be executed in an open loop system (not requiring feedback from one or more sensors 18), but the system 10 also may be executed in a closed loop system (which requires feedback from the one or more sensors 18).

The controller 12 can be coupled to the stimulator 14, which can be coupled to the one or more electrodes. In some instances, the coupling between the controller 12 and the stimulator 14 and/or the coupling between the stimulator 14 and the one or more electrodes 16 can be via a wired connection. In other instances, the coupling between the controller 12 and the stimulator 14 and/or the coupling between the stimulator 14 and the one or more electrodes 16 can be via a wireless connection. In still other instances, the coupling between the controller 12 and the stimulator 14 and/or the coupling between the stimulator 14 and the one or more electrodes 16 can be via a connection that is both wired and wireless. Similarly, in some instances, the one or more sensors 18 can be coupled to the controller 12 according to a wireless connection and/or a wired connection. Additionally, each element of the system 10 may have additional components to aid in the coupling that are not illustrated.

The controller 12 can include at least a non-transitory memory 2, a processor 4, and an input/output (I/O) 6. The non-transitory memory 2 can store machine executable instructions, which are executable by the processor 4 (for example, as shown in FIG. 2, the machine executable instructions can include receive input 22, define stimulation 24, and signal stimulator 26). In some instances, the non-transitory memory 2 and the processor 4 can be combined in a single hardware element (e.g., a microprocessor), but in other instances, the non-transitory memory 2 and the processor 4 can include at least partially distinct hardware elements. The I/O 6 can receive an input (e.g., based on a manual input, based on a predefined prescription, based on blood pressure recorded by the one or more sensors 18, or the like) and provide an output to the stimulator 14.

Upon receiving an output from the controller 12, the stimulator 14 can generate a stimulation signal. In some instances, the stimulator 14 can be part of the controller 12. In other instances, the stimulator 14 and the controller 12 can be at least partially distinct hardware elements. The stimulator 14 can be any device configured or programmed to generate the specified stimulation for application to Brodmann area 25. The stimulator 14 can be housed in the patient's body or outside the patient's body. One example of a stimulator 14 is a battery-powered, portable generator (positioned external to the patient's body). Another example of a stimulator 14 is an implantable generator (IPG) (at least a portion positioned subcutaneously). It will be appreciated that the stimulator 14 can include additional components to selectively configure the stimulation, such as an amplitude modulator (not shown).

The stimulation signal can be used to stimulate Brodmann area 25 and configured with parameters selected by the controller 12, and included in the output, with the goal of lowering blood pressure. The stimulation signal can be an electrical signal, a magnetic signal, a light signal, a heat signal, or any type of signal capable of neuromodulation in the brain. The stimulation signal can include an ON time, during which the stimulation is delivered, and an OFF time, during which the stimulation is not delivered. Using the example where the stimulation signal is an electrical signal (this example can be applied to the other stimulation modalities as well), during the ON time, the stimulation signal can include a waveform, such as a pulse train including balanced charge balanced biphasic pulses (e.g., a cathodic pulse followed by an equal and opposite anodic pulse or vice versa). The waveform can have one or more parameters, defined by the controller 12 and set by the stimulator 14, including at least one of a frequency, an intensity, a pulse width, a duration, and the like. The controller 12 can set and/or reset one or more of the parameters based on the input. As an example, the parameters can include a frequency from 20-50 Hz, an intensity from 6-10 mA, a pulse width from 0.2-0.5 ms, and a duration of application from 20-40 s. As another example, the parameters can include a frequency of 50 Hz, an intensity from 6-9 mA, a pulse width of 0.2 ms, and a duration of application from 20-40 s (e.g., an on time of 20-40 s and an off time until the next stimulation is delivered based on the input).

The stimulator 14 can provide the stimulation signal to one or more electrodes 16 to deliver the stimulation signal proximal to Brodmann area 25. The one or more electrodes 16 can be of any type configured to deliver the stimulation within the brain. For example, the one or more electrodes 16 can be cortical electrodes. As another example, the one or more electrodes can be deep brain stimulation (DBS) electrodes (e.g., depth electrodes). In some instances, the controller 12 can define in the output that the stimulator 14 should send the stimulation signal to certain electrodes of the one or more electrodes 16 to deliver the stimulation. For example, if the stimulation signal is delivered at regular intervals, the controller 12 can rotate the electrode delivering the stimulation so not to damage the brain tissue.

Upon delivery of the stimulation signal to Brodmann area 25, the patient's blood pressure can be lowered. One or more sensors 18 can be located in positions on the patient's body to detect this lowered blood pressure. As an example, the one or more sensors 18 can be external to the patient's body (like a sphygmomanometer). As another example, the one or more sensors 18 can be internal to the patient's body (e.g., proximal to an artery, like an intra-arterial sensor, artery cuff sensor, or the like).

The system 10 can be operated as an open loop system or a closed loop system. In the open loop system, the input to the controller is not based on output from the one or more sensors 18 fed back into the controller 12. For example, the input can be a manual input from a medical professional or the patient, which may be based on the output from the one or more sensors 18. As another example, the input can be delivered according to a predefined prescription (e.g., a number of stimulations every 24 hours). The prescription can be based on a titration to achieve the best results.

In the closed loop system, the input can be based on an output from the one or more sensors 18 being fed back into the controller 12. Upon receiving the input feedback, indicative of the patient's blood pressure, from the one or more sensors 18, the controller can determine whether a value associated with the blood pressure from the at least one sensor exceeds a threshold value (which is stored in the non-transitory memory 2). The threshold value can be an upper threshold and/or a lower threshold. The upper threshold can indicate a value of systolic blood pressure that indicates stimulation is necessary (e.g., an “on” switch). The lower threshold can indicate a value where the stimulation should not occur (e.g., an “off” switch).

For example, the upper threshold can be a systolic blood pressure greater than 120 mm Hg. As another example, the upper threshold can be a systolic blood pressure greater than 140 mm Hg. As a further example, the upper threshold can be a systolic blood pressure greater than 160 mm Hg. In some instances, the upper threshold can be variable based on previous stimulations and/or based on the systolic blood pressure of the patient. When the value associated with the blood pressure exceeds (or “is greater than”) the upper threshold value, a stimulation signal can be configured that will be applied to Brodmann area 25.

The lower threshold, for example, can be a systolic blood pressure less than 95 mm Hg. As another example, the lower threshold can be a systolic blood pressure less than 90 mm Hg. As a further example, the lower threshold can be a systolic blood pressure less than 88 mm Hg. In some instances, the lower threshold can be variable based on previous stimulations and/or based on the systolic blood pressure of the patient. When the value associated with the blood pressure is less than (or “exceeds”) the lower threshold value, no stimulation signal will be applied to Brodmann area 25.

The stimulator 16 can generate the stimulation signal according to the definition by the controller 12, which is applied proximal to Brodmann area 25 by the one or more electrodes 18. The one or more sensors 18 can detect the patient's blood pressure at a time after the stimulation and the closed loop system can start again.

The one or more sensors 18 can be configured to detect blood pressure according to a predefined schedule. In some instances, the one or more sensors 18 can detect blood pressure continuously at predetermined times. In other instances, the one or more sensors 18 can detect blood pressure at predetermined times for a period. In still other instances, the one or more sensors 18 can detect blood pressure and report the most recently detected blood pressure at input to the controller 12 when queried by the controller. In further instances, the one or more sensors 18 can detect blood pressure when queried by a user.

### IV. Methods

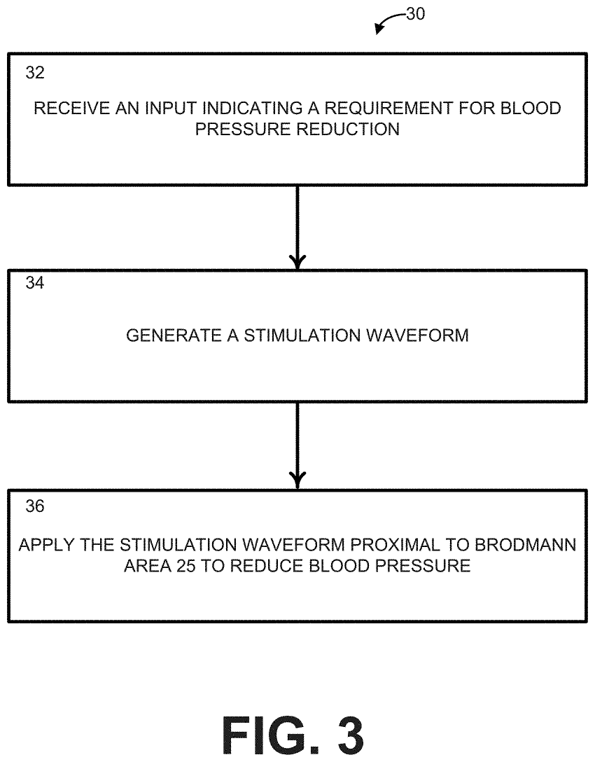

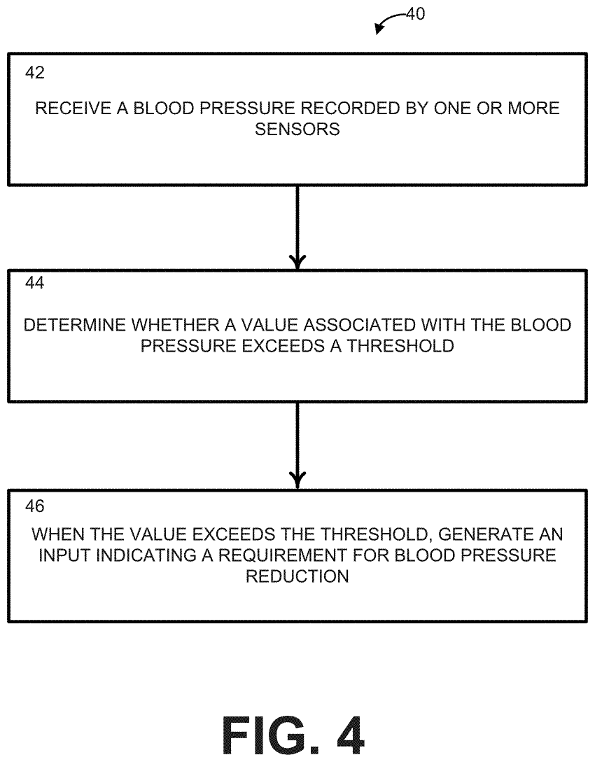

Another aspect of the present disclosure can include methods 30 and 40 (FIGS. 3 and 4) for lowering blood pressure by stimulating Brodmann area 25. The methods 30 and 40 are illustrated as process flow diagrams with flowchart illustrations. For purposes of simplicity, the methods 30 and 40 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 30 and 40.

Referring now to FIG. 3, illustrated is a process flow diagram of a method for lowering blood pressure by stimulating Brodmann area 25. The method 30 can be executed using the controller 12, the stimulator 14, and the electrode 16 shown in FIG. 1 and described above. The method 30 may be executed in an open loop system (not requiring feedback from the sensors 18 shown in FIG. 1), but also may be executed in a closed loop system (which requires feedback from the sensors 18).

At step 32, an input indicating a requirement for blood pressure reduction can be received (e.g., by controller 12). In the open loop system, the input is not based on output from sensors 18 fed back into the controller 12. For example, the input can be a manual input from a medical professional or the patient. As another example, the input can be delivered according to a predefined prescription (e.g., a number of stimulations every 24 hours). In the closed loop system, the input can be based on an output from the sensors 18 being fed back into the controller 12).

Based on the input, at step 34, a stimulation waveform can be generated (e.g., by stimulator 14). The stimulation waveform can be generated with parameters configured for blood pressure reduction. In some instances, the parameters can include at least one of a frequency, an intensity, a pulse width, and a duration, which can be configured according to an amount of blood pressure reduction desired. As an example, the parameters can include a frequency from 20-50 Hz, an intensity from 6-10 mA, a pulse width from 0.2-0.5 ms, and a duration of application from 20-40 s. As another example, the parameters can include a frequency of 50 Hz, an intensity from 6-9 mA, a pulse width of 0.2 ms, and a duration of application from 20-40 s (e.g., an on time of 20-40 s and an off time until the next stimulation is delivered based on the input). In either the open loop system or the closed loop system, the parameters can be preconfigured according to the predefined prescription to get the patient's blood pressure under what is defined as clinical hypertension (or another value, for example, if the blood pressure is very high and cannot be reduced safely to under clinical hypertension) and/or variable based on the input. At step 36, the stimulation waveform can be applied proximal to Brodmann area 25 (e.g., by one or more electrodes 16) to reduce the patient's blood pressure.

FIG. 4 illustrates an example method 40 for deciding whether to generate the input in the closed loop system example. At step 42, a blood pressure recorded by one or more sensors (e.g., sensor 18) is received (e.g., by controller 12). The blood pressure can be received after querying one or more of the sensors 18 (e.g., the query can instruct the one or more of the sensors 18 to detect the blood pressure, the query can instruct the one or more sensors 18 to send a blood pressure previously detected, or the like). However, the blood pressure can be received from the one or more sensors 18 at a predefined interval (e.g., more than one minute apart due to a residual effect of the stimulation).

At step 44, a determination can be made (e.g., by controller 12) as to whether a value associated with the blood pressure exceeds a threshold. The threshold value can be an upper threshold and/or a lower threshold. The upper threshold can indicate a value of systolic blood pressure that indicates stimulation is necessary (e.g., an “on” switch). The lower threshold can indicate a value where the stimulation should not occur (e.g., an “off” switch).

For example, the upper threshold can be a systolic blood pressure greater than 120 mm Hg. As another example, the upper threshold can be a systolic blood pressure greater than 140 mm Hg. As a further example, the upper threshold can be a systolic blood pressure greater than 160 mm Hg. In some instances, the upper threshold can be variable based on previous stimulations and/or based on the systolic blood pressure of the patient. When the value associated with the blood pressure exceeds (or “is greater than”) the upper threshold value, a stimulation signal can be configured that will be applied to Brodmann area 25.

The lower threshold, for example, can be a systolic blood pressure less than 95 mm Hg. As another example, the lower threshold can be a systolic blood pressure less than 90 mm Hg. As a further example, the lower threshold can be a systolic blood pressure less than 88 mm Hg. In some instances, the lower threshold can be variable based on previous stimulations and/or based on the systolic blood pressure of the patient. When the value associated with the blood pressure is less than (or “exceeds”) the lower threshold value, no stimulation signal will be applied to Brodmann area 25.

At step 46, when the value exceeds the threshold, generate (e.g., by controller 12) an input indicating a requirement for blood pressure reduction. The input can include a value indicative of the patient's blood pressure. For example, the input can reflect the detected systolic blood pressure. Based on the value, the controller 12 can adjust one or more of the parameters of the stimulation. For example, when the value associated with the blood pressure exceeds the threshold, the distance away from the threshold can be determined and the parameters can be adjusted based on the distance between the systolic blood pressure and the threshold.

### V. Experimental

The following experiment shows that Brodmann area 25 plays a role in lowering systolic blood pressure in humans and is a likely symptomatogenic site for peri-ictal hypotension. The following experimental results are shown for the purpose of illustration only and are not intended to limit the scope of the appended claims.

### Method

### Rationale

Several suprapontine brain structures governing blood pressure function have been identified, albeit inconsistently, in animals (orbitofrontal, cingulate, subcallosal, insular, hippocampal, amygdalar, temporal, and motor cortices) and in humans (orbitofrontal, insula, and anterior cingulated cortices). Therefore, these were the regions of interest for this study. The study aimed to identify the role of these structures in human blood pressure control using modern stereotactic techniques in patients undergoing invasive electroencephalogram (EEG) studies as a prelude to epilepsy surgery. All patients provided written informed consent as participants in a University Hospitals Cleveland Medical Center Institutional Review Board—approved research project evaluating the role of cortical structures in human respiratory and autonomic function.

### Patients and Clinical Setting

From Jun. 1, 2015, to Feb. 28, 2017, 12 consecutive patients with medically intractable epilepsy undergoing stereotactic EEG evaluations for epilepsy surgery in the Epilepsy Monitoring Unit at University Hospitals Cleveland Medical Center were studied prospectively. Inclusion criteria were patients 18 years or older who had electrodes implanted in one or more of the above-mentioned brain regions of interest and in whom direct cortical electrical stimulation was indicated for mapping of ictal onset or eloquent cortex regions. Electrodes surrounded by radiologically visible hemorrhage were excluded from study. The number and locations of depth electrodes were tailored according to the suspected location of the epileptogenic zone in each patient based on clinical history, semiology, neuroimaging, and noninvasive EEG.

### Procedure

Platinum-iridium depth electrodes measuring 1.1 mm in diameter and 2.5 mm in length, evenly spaced at 5-mm intervals, were implanted stereotactically using general anesthesia. Implantation trajectories were simulated using a software package (iPlan-Stereotaxy, version 2.6; Brainlab) based on recent 3-T magnetic resonance imaging (MRI) of the brain. Cranial computed tomography was performed within 24 hours after surgery. Using the iPlan-Stereotaxy software, postsurgical cranial computed tomography and presurgical brain MRI scans were superimposed for precise localization of single electrode contacts within the patient's presurgical MRI.

### Stimulation

Bedside cortical electrical stimulation was carried out using a stimulator (Ojemann; Integra Life Sciences) (bipolar and monopolar stimulation, 50 Hz and 0.2 milliseconds pulse width, with train durations of up to 30 seconds). Current intensity started at 1 mA to a maximum of 10 mA. These parameters were chosen for safety reasons because they are identical to those used for brain mapping for clinical purposes. If a seizure was induced, stimulation was discontinued. Resuscitation equipment was always kept in intimate proximity to the patient in case of need.

### Blood Pressure, Cardiac, Respiration, and EEG Monitoring

Beat-to-beat systolic (SAP), diastolic (DAP), and mean arterial pressure (MAP) were recorded using a continuous noninvasive arterial pressure monitor (Monitor 500; CNSystems Medizintechnik AG). Nasal air flow was recorded using a nasal thermistor (Thermocouple AirflowSensor; Pro-Tech). Arterial oxygen saturation and heart rate were monitored using pulse oximetry (Nellcor OxiMax N-600x; Covidien) and end-tidal carbon dioxide using a capnograph (Model 7900; Philips). Electroencephalogram and electrocardiogram were acquired using a diagnostic system (EEG-1200; Nihon Kohden) with a 256-channel amplifier. Blood pressure response was arbitrarily defined as a decrease or increase by more than 5 mmHg from the baseline mean during the stimulation period. The blood pressure response was only considered positive if there was a subsequent tendency to recover when stimulation was discontinued and when this response was consistently reproduced (during ≥5 sessions). Stimulation was initiated when SAP was within normal limits (100-125 mm Hg) and was immediately discontinued if it dropped either below 90 mmHg or by more than 25 mmHg from baseline. The EEG was closely scrutinized for stimulation-induced seizures and after-discharges during stimulation; if after-discharges were induced, that stimulation period was excluded from analysis.

### Data Analysis

A custom-developed graphical user interface (Matlab; MathWorks Inc) that included both signal processing and computational tools was used to automatically detect electrocardiogram R-wave, SAP, and DAP values as the maximum and minimum points between 2 consecutive R peaks. A series of four 5-minute consecutive epochs of artifact-free awake state rest recordings were identified as baseline. Twenty minutes of frequency-domain baroreflex sensitivity (BRS), blood pressure variability (BPV), and heart rate variability (HRV) values were averaged to calculate baseline values. Stimulation values were calculated from initiation of stimulus until heart rate and blood pressure returned to baseline levels.

Frequency-domain BRS was calculated as the average of the magnitude of the transfer function between oscillations of SAP and RR interval. Low-frequency (LF) range was defined between 0.04 and 0.15 Hz, and high frequency (HF) range was defined between 0.15 and 0.40 Hz. The ratio of LF to HF was used as a measure of sympathovagal balance. Total power (TP) for BRS18 and HRV13 was calculated as the sum of the LF and HF bands (TP=LF+HF) and was used to normalize frequency-domain values to correct for overall drops in total autonomic power. These normalized values were calculated by dividing the LF or HF band by TP and are reported as a percentage using the following equation:

Normalized Unit Value for HF=[HF/(LF+HF)]×100.

The evaluation of BRS is an established tool to assess autonomic control of the cardiovascular system. Changes in the characteristics of baroreflex function reflect alterations in autonomic control of the cardiovascular system. The quantitative measure of BRS is provided by the slope of the fitted line, and it is commonly expressed as the change in RR interval in ms per mmHg change in SAP (ms/mmHg). Calculated slope in sleep and awake states in healthy individuals is 9 to 12 ms/mmHg. An increase in SAP accompanied by a limited change in RR interval with a calculated slope lower than 3 ms/mm Hg identifies a pathological weak autonomic response.

### Statistical Analysis

All values are expressed as the mean (SEM). A paired-samples t test was conducted to compare stimulation averages with baseline values. Significance was set at 2-sided P<0.05. The strength of the linear association (correlation) between spontaneous SAP and RR intervals was assessed by Pearson product moment correlation coefficient r, and only those data sequences with r exceeding 0.7 were analyzed further.

### Results

Demographics and characteristics of the 12 patients (7 female; mean [SD] age, 44.25 [12.55] years) are summarized in Table 1.

TABLE 1
Patient and Epilepsy Characteristics and Blood Pressure Responses

| | | | Seizure | | Hypo- |
|---|---|---|---|---|---|
| Pa- | | | Dura- | | tensive |
| tient | | | tion, | | Re- |
| No. | Age | Sex | y | Seizure Semiology | sponses |
| 1 | 43 | F | 2 | Aura→dialeptic seizure→GTCS | No |
| 2 | 36 | F | 4 | Automotor seizure→right | No |
| | | | | versive seizure→GTCS | |
| 3 | 48 | M | 3 | Aura→dialeptic seizure | No |
| 4 | 39 | M | 10 | Automotor seizure→GTCS | No |
| 5 | 40 | M | 5 | Dialeptic seizure→GTCS | No |
| 6 | 66 | M | 30 | Automotor seizure→GTCS | No |
| 7 | 20 | F | 14 | Apnea seizure→right versive | Yes |
| | | | | seizure→GTCS | |
| 8 | 32 | F | 25 | Hypnopompic seizure→right | Yes |
| | | | | versive seizure→GTCS | |
| 9 | 26 | M | 8 | Automotor seizure | Yes |
| 10 | 49 | F | 3 | Automotor seizure | No |
| 11 | 69 | F | 44 | Abdominal aura →automotor | No |
| | | | | seizure→GTCS | |
| 12 | 63 | F | 50 | Asymmetric tonic | Yes |
| | | | | seizure→GTCS | |

Abbreviations: F, female; GTCS, generated tonic-clonic seizure; M, male.

Patient 12 had essential hypertension treated with amlodipine besylate (5 mg/d) that was continued during the evaluation. The rest of the patients did not have any cardiorespiratory comorbidity and were not taking any cardiovascular medications. At the time of stimulation, patient 1 was taking his regular dosage of lacosamide (200 mg/d) and topiramate patient 12 was taking levetiracetam (4500 mg/d), vimpat (600 mg/d), and clobazam (30 mg/d). The remaining patients were off antiepileptic medications as a part of the clinical protocol aimed at capturing seizures to localize the epileptogenic zone. In total, 1084 electrode contacts were implanted based on the surgical hypothesis in each case. Of these, 544 electrodes were implanted in our regions of interest, including 43 amygdala, 87 hippocampus head and body, 16 insular, 31 orbitofrontal, 31 temporopolar, 296 lateral temporal, 4 basal temporal, 13 anterior cingulate, 9 subcallosal (Brodmann area 25), and 14 posterior cingulated neocortex. Of 544 electrodes, 126 were stimulated according to the study protocol, including 23 amygdala, 17 hippocampus head, 8 anterior insula, 16 orbitofrontal, 12 temporopolar, 24 lateral temporal, 2 basal temporal, 13 anterior cingulate, 9 subcallosal neocortex (Brodmann area 25), and 2 posterior cingulate. The rest of the electrodes (540 of 1084) were placed either in white matter or in gray matter outside our regions of interest.

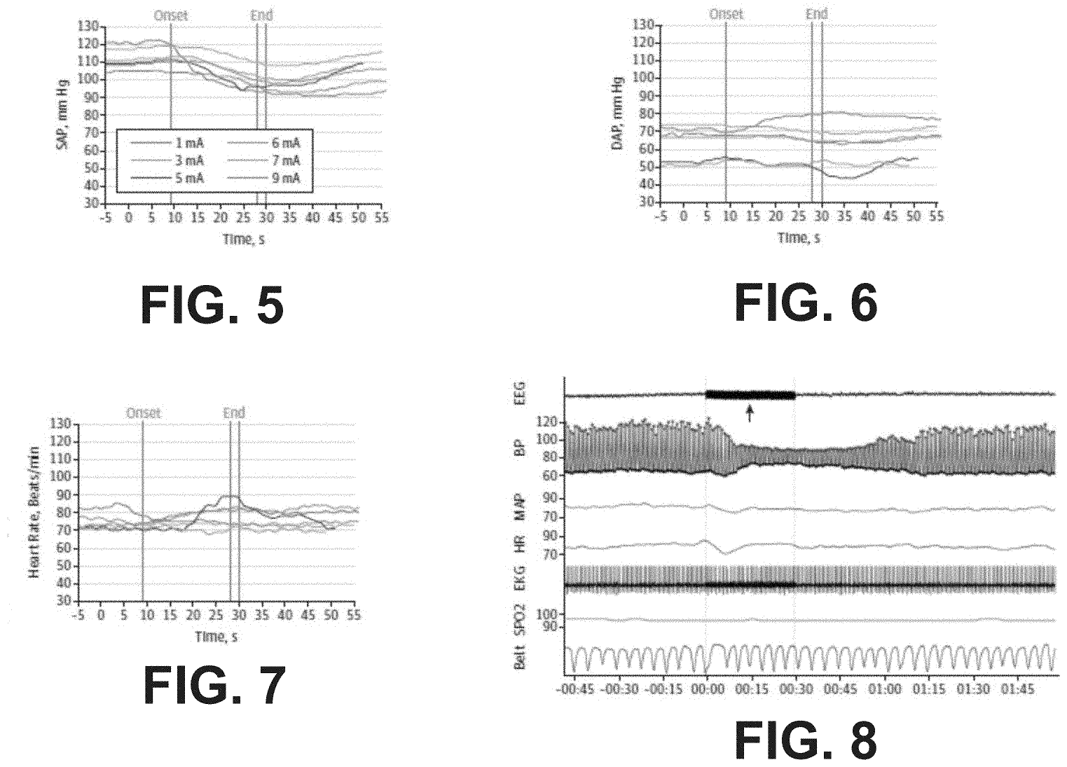

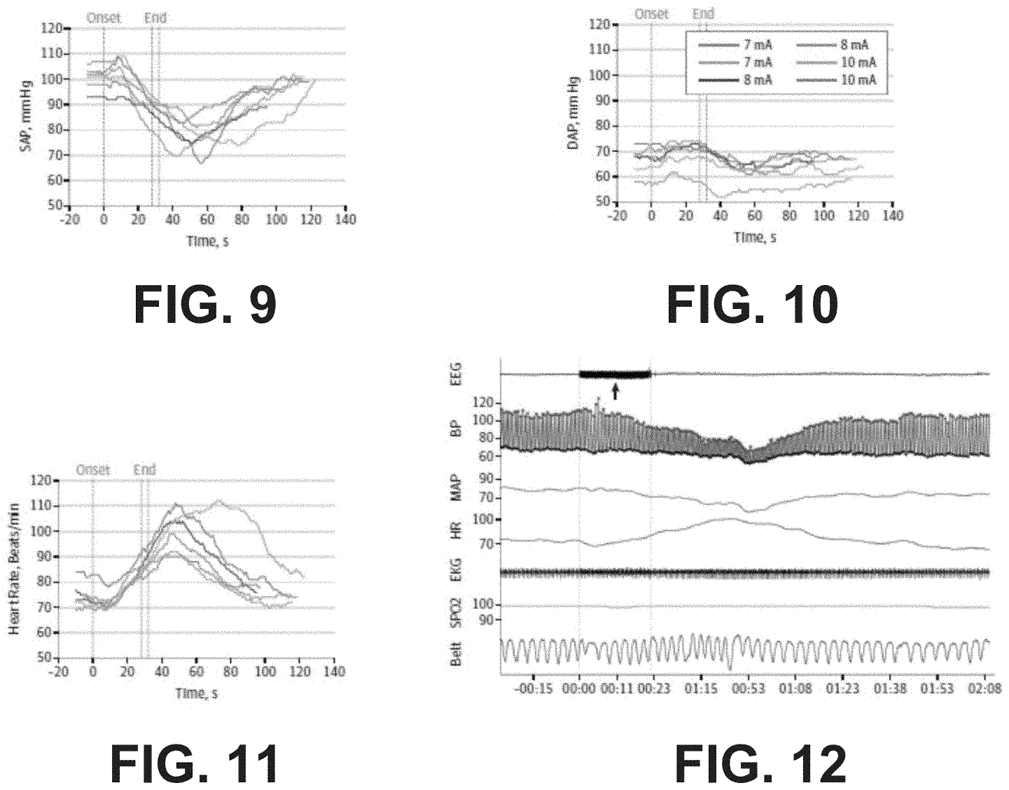

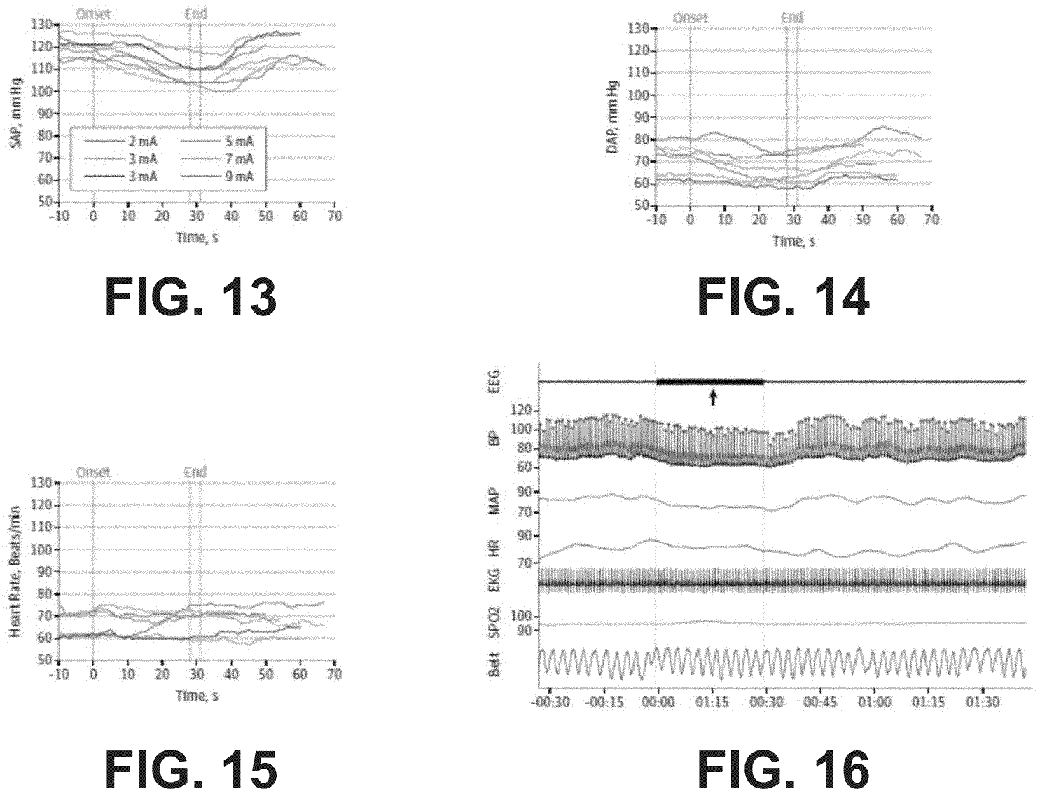

Results of stimulation of electrodes placed in Brodmann area 25 in patients 7, 8, and 8 are shown in FIGS. 5-16. Stimulation in all electrodes placed in Brodmann area 25 in patients 7, 8, 9, and 12 (in whom 9 electrodes [7 left and 2 right] produced striking systolic hypotensive changes) resulted in rapid and consistently reproducible decreases in SAP, with a mean (SEM) drop of 15 (10-42) mmHg. The SAP decreases appeared after a mean (SEM) latency of 8.5 (1-14) seconds. At times, the fall in SAP was preceded by a slight rise. Once stimulation was discontinued, SAP began to increase within a mean (SEM) of 12 (1-47) seconds. The DAP did not change concurrently with SAP, resulting in a consistent narrowing of pulse pressure in all patients. Heart rate responses differed. In patient 8, heart rate increased accordingly with SAP. On the other hand, in patients 7, 9, and 12, heart rate did not significantly change. Arterial oxygen saturation and end-tidal carbon dioxide did not change at any time during or after stimulation. Frequency-domain analysis of BRS, BPV, and HRV comparing baseline with the stimulation period and baroreflex slope was performed in patients 7, 8, and 9 and showed a mixed picture.

During some of the Brodmann area 25 stimulation sessions, brief after-discharges were induced, although there were no differences in blood pressure responses when the after discharges were induced or not. However, stimulations with after-discharges were excluded to ensure that blood pressure responses were being produced exclusively by Brodmann area 25 stimulation and not by after-discharges in other brain areas.

Recorded seizures were analyzed in those patients in whom we hypotensive responses were found to specifically look for spontaneous peri-ictal hypotensive changes and for correlation of seizure discharges in Brodmann area 25 with hypotension. Patient 7 did not have hypotensive changes with the single seizure that was recorded; the Brodmann area 25 electrode was involved in the seizure, although the seizure discharge was widespread at that point. Patient 8 had no blood pressure recordings during seizures. Patient 9 had no seizures recorded during intracranial EEG monitoring with blood pressure recordings. However, he previously had 3 complex partial seizures with oral automatisms recorded with surface EEG and continuous blood pressure monitoring. Two of these had ictal and post-ictal hypotension. Patient 12 had asymmetric tonic seizures lasting for less than 10 seconds in which blood pressure did not change and where the seizure did not involve Brodmann area 25.

No significant blood pressure responses were noted after stimulation of amygdala, hippocampus, and insular, orbitofrontal, temporopolar, lateral temporal, basal temporal, anterior cingulate, and posterior cingulate neocortex. Central apnea induced by stimulation was observed in temporal lobe structures and reported separately. Apnea was not associated with blood pressure responses.

From the above description, those skilled in the art will perceive improvements, changes and modifications. Such improvements, changes and modifications are within the skill of one in the art and are intended to be covered by the appended claims.

The following is claimed:

1. A system comprising:
at least one sensor configured to detect blood pressure;
a controller configured to:
receive the blood pressure from the at least one sensor;
determine whether a value associated with the blood pressure from the at least one sensor exceeds a threshold value; and
when the value associated with the blood pressure from the at least one sensor exceeds the threshold value, signal a stimulator to generate a stimulation waveform;
the stimulator configured to generate the stimulation waveform; and
at least one electrode configured to be located proximal to Brodmann area 25 and to deliver the stimulation waveform to Brodmann area 25 to reduce the blood pressure.

2. The system of claim 1, wherein the at least one sensor is configured to be located proximal to an artery within a patient's body to detect the blood pressure.

3. The system of claim 1, wherein the at least one electrode comprises at least one depth electrode configured for implantation in a patient's brain.

4. The system of claim 1, wherein the stimulation waveform is bipolar and biphasic, comprising a frequency from 20-50 Hz, an intensity from 6-10 mA, a pulse width from 0.2-0.5 ms, and a duration of application from 10-40 s.

5. The system of claim 1, wherein the at least one sensor is configured to detect the blood pressure continuously at predetermined times and send the blood pressure detected at the predetermined times to the controller to determine whether the value associated with the blood pressure exceeds the threshold value.

6. The system of claim 1, wherein the at least one sensor is configured to detect the blood pressure in response to being queried by a user.

7. The system of claim 1, wherein the controller comprises a memory storing the threshold value and instructions and a processor to execute the instructions to determine whether the value associated with the blood pressure from the at least one sensor exceeds the threshold value.

8. The system of claim 1, wherein a parameter of the stimulation is adjustable by the controller based on the value associated with the blood pressure.

9. A method comprising:
receiving, by a controller comprising a processor, an input indicating a requirement for blood pressure reduction;
signaling, by the controller, a stimulator to generate a stimulation waveform with parameters configured for the blood pressure reduction;
generating, by the stimulator, the stimulation waveform with the parameters configured for the blood pressure reduction; and
applying, by at least one electrode located proximal to Brodmann area 25, the stimulation waveform proximal to Brodmann area 25 to reduce the blood pressure.

10. The method of claim 9, wherein the stimulation waveform is bipolar and biphasic and the parameters comprise a frequency from 20-50 Hz, an intensity from 6-10 mA, a pulse width from 0.2-0.5 ms, and a duration of application from 10-40 s.

11. The method of claim 9, further wherein the receiving further comprises:
receiving a blood pressure recorded by at least one sensor;
determining whether a value associated with the blood pressure from the at least one sensor exceeds a threshold value; and
when the value associated with the blood pressure from the at least one sensor exceeds the threshold value, generating the input indicating the requirement for blood pressure reduction.

12. The method of claim 11, further comprising querying the at least one sensor to detect the blood pressure.

13. The method of claim 11, wherein the blood pressure is detected continuously by the at least one sensor at predetermined times at least 1 minute apart because the stimulation exhibits a residual effect.

14. The method of claim 11, further comprising adjusting, by the controller, one or more of the parameters based on the value associated with the blood pressure.

15. The method of claim 11, further comprising, when the value associated with the blood pressure exceeds the threshold, determining how far the value associated with the blood pressure differs from the threshold.

16. The method of claim 15, further comprising adjusting the one or more parameters based on how far the value associated with the blood pressure differs from the threshold.

17. The method of claim 16, wherein the one or more parameters are designed to reduce the blood pressure by at least 20 points systolic.

18. A controller comprising:
a memory storing instructions; and
a processor configured to access the memory and execute the instructions to:
receive an input indicating a requirement for blood pressure reduction; and
signal a stimulator to generate a stimulation waveform to be delivered by at least one electrode located proximal to Brodmann area 25,
wherein the stimulation waveform is configured to be delivered to Brodmann area 25 by the at least one electrode to reduce the blood pressure.

19. The controller of claim 18, wherein the input is derived by:
receiving a blood pressure recorded by at least one sensor;
determining whether a value associated with the blood pressure from the at least one sensor exceeds a threshold value; and
when the value associated with the blood pressure from the at least one sensor exceeds the threshold value, generating the input indicating the requirement for blood pressure reduction.

20. The controller of claim 19, wherein the blood pressure recorded by the at least one sensor is received continuously at predetermined times.
